(19) United States
(12) Patent Application Publication
(10) Pub. No.: US 2006/0224117 A1
(43) Pub. Date: Oct. 5, 2006

(54) System for administering an injectable product

(76) Inventors: Edgar Hommann, Grossaffoltern (CH); Benjamin Scherer, Uster (CH); Christoph Rindlisbacher, Boll (CH); Ian Thompson, Burgdorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/367,043

(22) Filed: Mar. 2, 2006

Related U.S. Application Data
(63) Continuation of application No. PCT/CH04/00551, filed on Sep. 1, 2004.

(30) Foreign Application Priority Data
Sep. 3, 2003 (DE) 103 40 584.4

(51) Int. Cl. A61M 5/20

(52) U.S. Cl. 604/136

(57) ABSTRACT

A system for administering an injectable product including an injection syringe carrying a needle, a drive unit and a housing for accommodating the injection syringe and the drive unit, wherein the drive unit moves the injection syringe relative to the housing, and wherein, in some embodiments, a retractor mechanism is provided to retract the needle into the housing after the product is administered.

### CROSS-REFERENCE TO RELATED APPLICATION(S)

This application is a continuation of International Application No. PCT/CH2004/000551, filed on Sep. 1, 2004, which claims priority to German Application No. 103 40 584.4, filed on Sep. 3, 2003, the contents of which are incorporated in their entirety by reference herein.

### BACKGROUND

The present invention relates to devices for administering, delivering, dispensing or injecting substances, and to methods of making and using them. More particularly, the present invention relates to a system, apparatus or combination of component mechanisms or devices for administering an injectable product, comprising an injection syringe, a drive unit and a housing for accommodating the injection syringe and the drive unit.

Various medical and therapeutic applications require an injectable product to be administered. The simplest possible way of administering the product is to use a conventional injection syringe. The product is already accommodated in the chamber of the syringe or has to be drawn into it. A body tissue is then pierced by hand using an injection needle carried by the injection syringe, which is firmly seated on the injection syringe, and the product is then administered from the injection syringe by pushing forward a feed member, such as a plunger rod, by hand. Administering the product with an injection syringe tends to require a certain amount of practical experience to avoid pain when inserting the needle and to ensure that the product is administered at a correct rate and in a desired dose. Using an injection needle also poses a risk of injury due to the exposed needle.

Various types of administering devices have been developed with a view to simplifying the task of administering an injectable product so that even a lay person or patient can self-administer it, for example. Such administering devices generally have a chamber for accommodating the injectable product and an administering mechanism. The injectable product may be accommodated directly by the chamber or an ampoule accommodating the product may be inserted in the chamber. The administering device may have a drive means for driving a feed member in order to administer or deliver the product from the chamber by pushing the feed member forward. Administering devices are also known which have a piercing aid for automatically piercing a body tissue with an injection needle of the administering device.

Various types of systems are also known, which combine a conventional injection syringe and an administering device for automatically administering the injectable product from the syringe. A metering mechanism may also be provided, to enable a pre-settable dose to be dispensed from the injection syringe. Such a system simplifies the task of administering the injectable product from the injection syringe. However, it is also necessary to pierce the body tissue with the injection needle by hand and remove it again once the product has been administered, running the risk of injury from the exposed needle. A needle protector cap may be provided over the injection needle. However, it has to be placed over the needle by hand before and after administering. These manipulations delay the process of administering an injectable product and also pose a risk of injury by the injection needle.

### SUMMARY

An objective of the present invention is to provide a system for administering an injectable product from an injection syringe, which enables the product to be administered easily and reliably, reduces the risk of injury during the administering process and is inexpensive.

This objective is addressed by providing, in accordance with the present invention, a system for administering an injectable product including an injection syringe carrying a needle, a drive unit and a housing for accommodating the injection syringe and the drive unit, wherein the drive unit moves the injection syringe relative to the housing, and wherein, in some embodiments, a retractor mechanism is provided to retract the needle into the housing after the product is administered.

In one embodiment, the present invention comprises a device for administering an injectable product, the device comprising an injection syringe, from which the injectable product is delivered through an injection needle carried by the injection syringe, a drive unit, and a housing for accommodating the injection syringe and the drive unit, wherein the drive unit moves the injection syringe relative to the housing.

In one embodiment, the present invention comprises a system for administering an injectable product comprising an injection syringe, a drive unit and a housing for accommodating the injection syringe and the drive unit. The injection syringe has a syringe housing, in which a product chamber for the injectable product is housed. The product chamber is closed off at one end by a stopper which can be moved relative to the syringe housing. Adjoining the syringe housing and the product chamber is an injection needle, which has a fluid connection to the product chamber. By pushing a feed member forward, such as a plunger rod which drives the stopper inside the product chamber, the product can be administered via the injection needle from the injection syringe. The drive unit preferably has a mechanical drive but may also have some other drive, such as a gas-operated drive, for example. As proposed by the invention, the drive unit is designed so that it drives the injection syringe in the longitudinal direction of the housing relative to the housing, until the injection needle of the injection syringe projects out from the housing. In other words, the injection syringe is pushed by the drive unit as a unit inside the housing so that the injection needle of the injection syringe can pierce a body tissue, advantageously covering the total piercing depth necessary. In some embodiments. the movement of the injection syringe is preferably effected in the forward stroke direction of the feed member of the syringe provided as a means of administering the product, and hence in the administering direction.

With the aid of an administering system of the type in accordance with the invention, it is possible to use a conventional injection syringe for administering an injectable product, which can be produced inexpensively on a mass scale. The risk of injury when using such an injection syringe can be reduced because the piercing action of the injection needle of the injection syringe is effected automatically, directly from the housing of the system, in which case the housing assumes the function of protecting the needle. The needle is able to pierce a body tissue directly from the housing by placing the housing on a tissue surface, thereby ruling out any risk of injury.

The injection syringe, the drive unit and the housing of the administering system of the present invention are advantageously provided as separate components, which make up a device or system for administering an injectable product once assembled. The individual components of the system form a kit for an administering device. Using finished components makes it easy to cater for specific requirements in that only individual components have to or may be changed. For example, the same drive unit can be used for ready-made injection syringes of different types or different drive units may be used with a specific injection syringe.

In a preferred embodiment of the administering system of the present invention, a retractor mechanism for retracting the injection needle into the interior of the housing is provided. In one preferred embodiment, the retractor mechanism is disposed in the injection syringe and pulls the injection needle back into the interior of the syringe housing. Once an injectable product has been administered, the retractor mechanism pulls the injection needle out of the tissue and directly back into the housing. Accordingly, at no time during the administration process is the injection needle accessible to a user, thereby ruling out any risk of injury. An injection syringe with such a retractor mechanism can be manufactured in large numbers on a mass scale. A syringe of this type may be obtained from NMT New Medical Technology, for example.

It is advantageous if the drive unit, in addition to providing the drive or motive force for the injection syringe, also serves as a drive for the feed member of the injection syringe, by means of which the feed member is pushed forward relative to the syringe housing in order to administer an injectable product. A simple plunger rod may be used as the feed member, which pushes a stopper forward inside a product chamber of the injection syringe, in the manner which traditionally takes place by hand. To this end, the drive unit may have a drive member, which, during a first step of the administering process, drives the injection syringe relative to the housing of the administering system and, in a second step, drives the feed member of the injection syringe relative to the syringe housing. This will produce an automatic piercing action by the needle and then dispense the product from the injection syringe.

In one advantageous embodiment, the retractor mechanism is released by the drive unit. The retractor mechanism can be released by a stop of the feed member, for example, once the injectable product has been successfully administered from the injection syringe. The feed member may hit a stop, for example at the end of the forward stroke in the product chamber, which constitutes a termination of the chamber and thus releases the retractor mechanism. When the feed member is moved in the forward stroke direction with the aid of the drive unit, the retractor mechanism is released by means of the drive unit. In this embodiment of the administering system of the present invention, the drive unit automatically moves the injection syringe forward in order to insert the injection needle, moves the feed member forward in order to dispense the product from the injection syringe and pulls the injection needle back into the housing. The injection needle is therefore not accessible at any time during the administering process.

The administering system of the present invention may incorporate a release mechanism which releases the drive unit and, in some preferred embodiments, also the retractor mechanism. To this end, the drive mechanism and the retractor mechanism are held in a pre-tensioned state, which can be released by operating the release mechanism so that the drive unit and the retractor mechanism can fulfil their function by means of the biasing force. In some embodiments, the release mechanism is designed so that the drive mechanism is pre-tensioned by means of a spring disposed, for example, between the drive member and a housing part, and fixed relative to the housing part in the pre-tensioned state. For releasing purposes, the drive member is released by the release mechanism relative to the housing so that it moves in the forward stroke direction relative to the housing part, thereby driving the feed member of the injection syringe and finally the retractor mechanism, which may be released in the manner described above.

The release mechanism may preferably take the form of two housing parts of the housing which are able to move relative to one another. A first housing part is used as a means of fixing the drive unit in a pre-tensioned state and the second housing part is used as a means of releasing the release mechanism, i.e., terminating the pre-tensioned state. To this end, the second housing part is pushed relative to the first housing part until the drive unit or the drive member is released. A second housing part lying close to the tissue is pushed in the direction opposite the forward direction relative to a first housing part lying remote from the tissue. If the administering system is held by the first housing part, for example, and placed on a surface of a tissue in readiness for the administering process, the second housing part can be pushed relative to the first housing part in the direction opposite the forward direction by pressing on the administering system, thereby triggering the drive unit. This obviates the need for a separate hand grip for releasing the administering system. In order to administer the injectable product, the system is placed on a tissue surface and pressed. The entire administering procedure is then run automatically due to the co-operation of the drive unit and the retractor mechanism.

### BRIEF DESCRIPTION OF THE DRAWINGS

FIG. 1 is shows a longitudinal section through components of an administering system in accordance with the present invention,

FIG. 2 is a longitudinal section through an assembled administering system in accordance with the present invention in a locked position,

FIG. 3 is a longitudinal section through the administering system illustrated in FIG. 2 in a released position,

FIG. 4 is a longitudinal section through the administering system illustrated in FIG. 2 in an injecting position,

FIG. 5 is a longitudinal section through the administering system illustrated in FIG. 2 in a dispensed position, and

FIG. 6 is a longitudinal section through the administering system illustrated in FIG. 2 with the needle in a retracted position.

### DETAILED DESCRIPTION

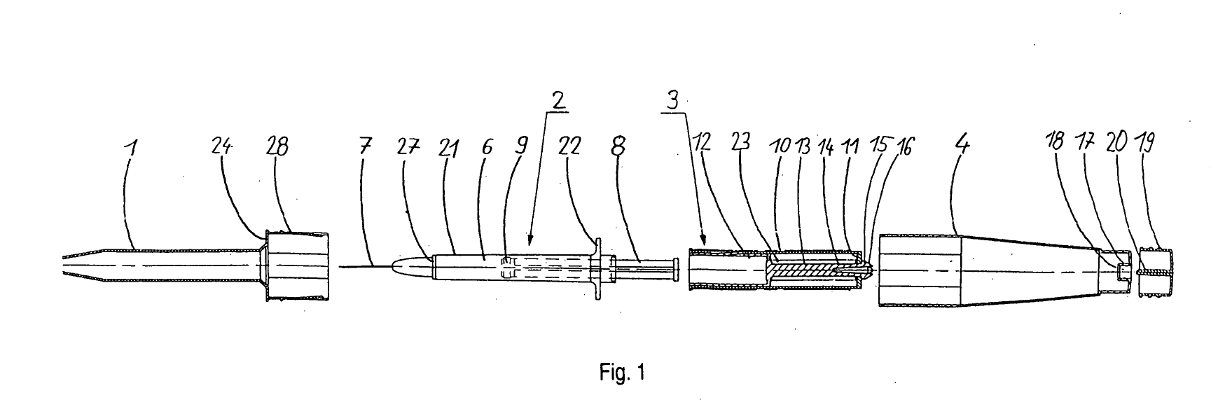

FIG. 1 illustrates the individual components of an administering system or device in accordance with the present invention. The administering system comprises a first front housing part 1, an injection needle 2, a drive unit 3 and a second rear housing part 4. The first housing part 1 has a generally cylindrical, sleeve-shaped front region with a slim or small diameter for accommodating the injection syringe 2. The rear region of the first housing part 1 is of a fork-shaped design. At the end of the sleeve-shaped front region, two oppositely lying webs 24 stand vertically or transversely outwardly from the peripheral surface, from which two arms 28 project generally in the direction of or parallel to the longitudinal axis of the sleeve-shaped region (and the device or system overall). The distance between the oppositely lying arms 28 is larger than the diameter of the sleeve-shaped region. The rear fork-shaped region of the first housing part 1 serves as a holder or stop for the injection syringe 2. A conventional syringe is used as the injection syringe, for example of the type manufactured by NMT New Medical Technology. The injection syringe has a syringe housing 21 with a chamber 6 accommodated therein for a fluid product or substance and an injection needle 7 carried at the front end. At the rear end, a feed member in the form of a plunger rod 8 projects out from the injection syringe 2. The rod 8 is displaceable in the longitudinal direction relative to the syringe and is used as a means of pushing a stopper 9 forward inside the chamber 6. Mounted at the rear end of the syringe housing 21 is a projecting collar 22.

The drive unit 3 has a sleeve 10, which is open at the front end and has a terminal wall 11 with a central orifice at the rear end. Mounted in the sleeve 10 is a drive member 12, which is displaceable in the longitudinal direction relative to the sleeve 10. The drive member 12 is of a sleeve-shaped design in a front region and the external peripheral surface of the sleeve region lies against the internal peripheral surface of the sleeve 10. Adjoining the front sleeve-shaped region is a rod 13 extending centrally along the longitudinal axis of the sleeve 10. At the rear end, the rod 13 splits into two oppositely lying wings 14, which have projections 15 extending outwards from the peripheral surface of the rod 13 at their ends and oblique terminal surfaces 16 converging to a point on the longitudinal axis. There is a gap between the oppositely lying wings 14 so that the wings 14 can be bent radially inwards by applying a force, i.e., can be bent towards one another. The end of the rod 13 of the drive member 12 projects through the central orifice of the terminal wall 11 and the projections 15 move to lie against the external face of the terminal wall 11. In this position, the drive member 12 is not displaceable in the forward stroke direction relative to the sleeve 10. The drive member 12 is held in a pre-tensioned state by means of a spring 23, with the projections 15 lying against the terminal wall 11. To this end, the spring 23 is inserted between the sleeve-shaped region of the drive member 12 and the internal face of the terminal wall 11 of the sleeve 10 around the rod 13.

The second rear housing part 4 is of a sleeve-shaped design so that it can accommodate the drive unit and adjoins the complementary region of the first housing part 1 having a wide diameter in a positive fit by its front end. The rear end has a coaxially disposed, inwardly directed sleeve-shaped extension 17, which is disposed around a central orifice 18. A positively fitting locking cap 19 can be placed on the rear end of the second housing part 4. The locking cap 19 has a pin 20 directed inwards in the direction of the second housing part 4. When in the state placed on the housing part 4, the pin 20 projects through the orifice 18 of the housing part 4. As may be seen and understood from FIG. 1, the system for administering an injectable product in accordance with the present invention is a modular system.

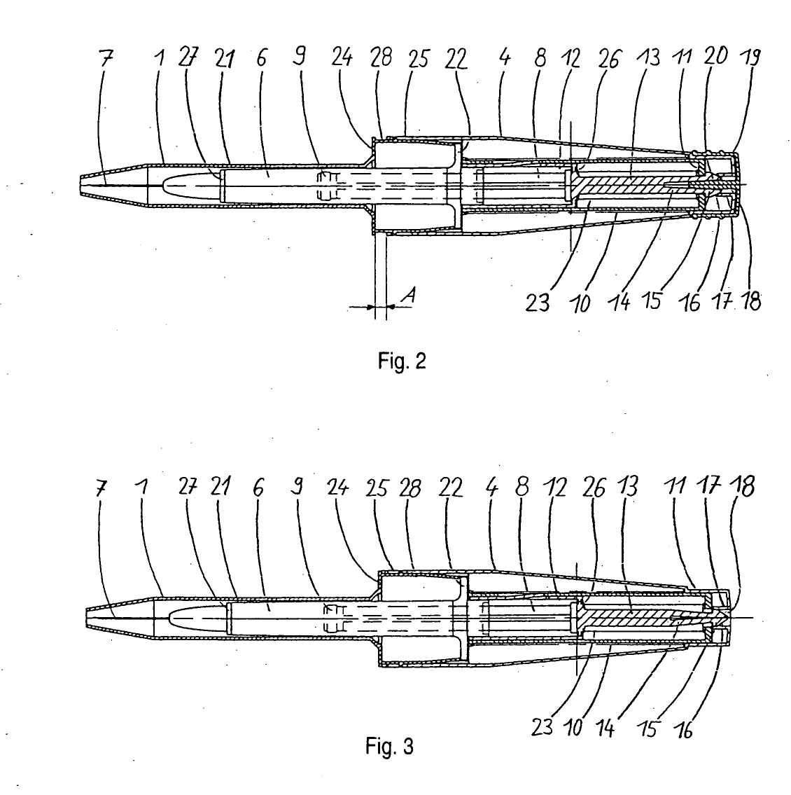

FIG. 2 illustrates the individual components of the administering system illustrated in FIG. 1, but in the assembled state. The injection syringe 2 with the injection needle 7 is accommodated in the interior of the front region of the first housing part 1 with the slim diameter. The injection needle 7 is therefore surrounded by the first housing part 1. The injection syringe 2 is inserted in the first housing part 1 so far in the forward or delivery stroke direction that the collar 22 sits against the end of the arms 28 and the fork-shaped region of the first housing part 1. The arms 28 are bent slightly inwards at their ends and have retaining means at their ends for accommodating the edge of the collar 22. In this locked position, the injection syringe 2 is not able to move farther in the forward stroke direction relative to the first housing part 1. The plunger rod 8 extending to the rear out of the injection syringe 2 projects into the interior of the sleeve-shaped front region of the drive member 12 and abuts with the end face of the rod 13. In this state, the front end of the sleeve 10 and the front end of the drive member 12 abut with the collar 22 of the syringe housing 21. The second rear housing part 4 is placed in a positive fit on the fork-shaped region of the first housing part 1 over the drive unit 3 and over the rear part of the injection syringe 2.

The locking cap 19 is placed on the rear end of the second housing part 4. Accordingly, the pin 20 of the locking cap 19 extends through the central orifice 18 of the second housing part 4 and into the gap between the wings 14 of the rod 13 of the drive member 12. The pin 20 of the locking cap 19 prevents the wings from bending inwards. The front ends of the sleeve-shaped extension 17 abut with the oblique terminal surfaces 16 of the rod 13.

The front end of the second housing part 4, which is pushed onto the fork-shaped region of the first housing part 1, terminates at a distance A in front of the webs 24. The outwardly directed webs 24 on the first housing part 1 form a stop for the second housing part 4, which prevents the second housing part 4 from moving in the forward stroke direction beyond the webs 24 when the housing parts 1 and 4 are displaced relative to one another.

In the position illustrated in FIG. 2, the second housing part 4 is prevented from being moved forwards relative to the first housing part 1 because the front end of the sleeve-shaped extension 17 is supported against the oblique terminal faces 16 at the rear end of the rod 13, the projections 15 on the drive member 12 are supported against the external face of the terminal wall 11 of the sleeve 10, the front end of the sleeve 10 is supported against the collar 22 of the injection syringe 2, and the collar 22 is supported against the arms 28 of the first housing part 1. All in all, therefore, the individual components of the administering system are disposed in a fixed arrangement with respect to one another. A catch mechanism or a guide groove 25 may be provided in addition, which prevents or makes it more difficult for the first housing part 1 and the second housing part 4 to come apart from one another by pulling the second housing part 4 relative to the first housing part 1 in the direction opposite the forward stroke direction. In this position, the administering system is in a locked position because the fitted locking cap 19 prevents the wings 14 from being compressed and thus prevents a movement of the individual elements with respect to one another.

The inwardly projecting sleeve-shaped extension 17, the wings 14 with the oblique terminal faces 16 and the projections 15 as well as the central orifice in the terminal wall 11 of the drive unit 3 and the pre-tensioned spring 23 constitute a release mechanism, by means of which the drive unit 3 and the retractor mechanism of the injection syringe 2 can be released.

FIG. 3 illustrates the administering system in a released position. The locking cap 19 has been taken off the second housing part 4, as a result of which the pin 20 has been removed from the gap between the wings 14 of the rod 13. The second housing part 4 has been pushed forward out of the locked position relative to the first housing part 1 by the distance A in the forward stroke direction and sits with the front end on the webs 24. As the second housing part 4 is moved forward in the forward stroke direction relative to the first housing part 1, the second housing part 4 is also pushed forward in the forward stroke direction relative to the injection syringe 2 and the drive unit 3. Accordingly, the front end of the inwardly projecting sleeve-shaped extension 17 is pushed beyond the oblique terminal faces 16 of the wings 14 so that the wings 14 are compressed inwards. This reduces the diameter of the outer edges of the projections 15 on the wings 14 to the degree that the projections 15 no longer lie against the terminal wall 11 of the sleeve 10 but fit through the central orifice in the terminal wall 11. The drive member 12 can therefore be displaced in the forward stroke direction relative to the sleeve 10 of the drive unit 3.

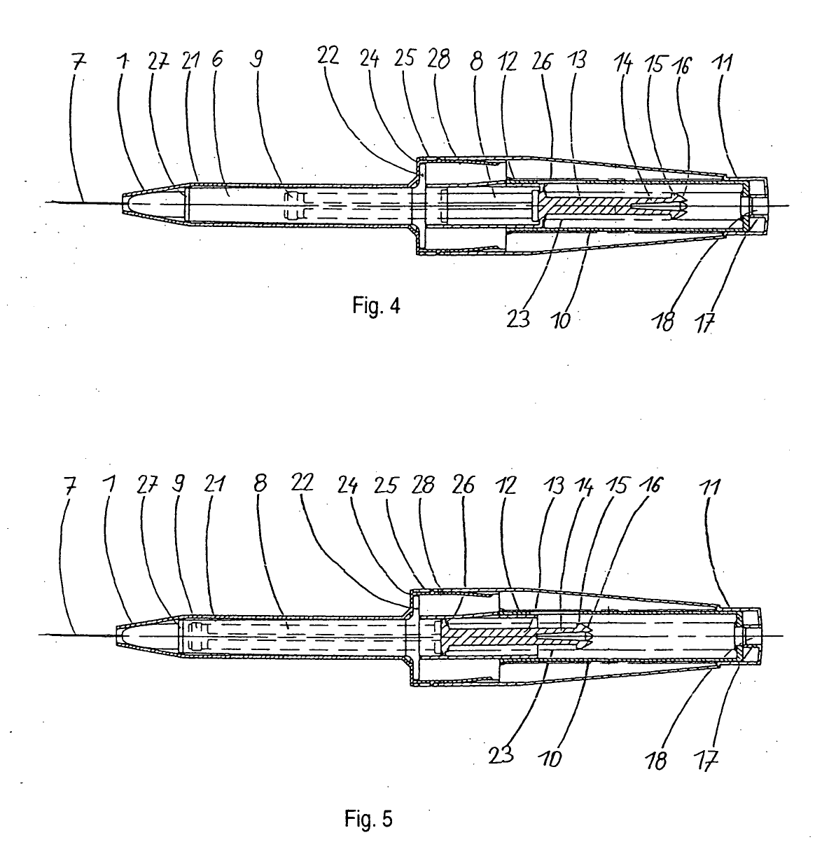

FIG. 4 shows the administering system in an injecting position in which the injection needle 7 is projecting forwardly or extending out of the first housing part 1. As the drive member 12 is released, it is displaced relative to the sleeve 10 of the drive unit 3 by the pre-tensioning of the spring 23. Since the front end of the sleeve-shaped region of the drive member 12 lies against the collar 22 of the injection syringe 2, the driving force of the spring 23 is transmitted to the collar 22. The collar 22 can therefore sit against the ends of the arms 28 so that it slips under the action of a force from the arms 28 and is able to move between the arms 28. As a result of the forward stroke of the drive member 12, the collar 22 is therefore released from its abutment on the arms 28 of the first housing 1 and is pushed relative to the first housing 1 until it abuts with the webs 24. The injection syringe 2 is therefore pushed or moved by the forward stroke movement of the drive member 12 relative to the first housing part 1 in the forward stroke direction and the injection needle 7 moves out beyond the front end of the housing part 1.

A control cam may preferably provided as a means of releasing the abutment of the collar 22 with the arms 28 on the second housing part 4. As the first housing part 1 and the second housing part 4 move relative to one another, the arms 28 are widened with the aid of the control cam so that the collar 22 can be moved in the forward stroke direction relative to the first and second housing part. The control cam may take the form of a surface structure, for example, on the internal peripheral face of the second housing part 4, for example. The external faces of the arms 28 may lie against the control cam via a contact element or directly, for example, and are released from an inwardly directed pre-tensioned position by the curved contour.

FIG. 5 illustrates the administering system in a dispensed position in which the plunger rod 8 of the injection syringe 2 has been pushed forward in the forward stroke direction relative to the syringe housing 21 and has pushed the stopper 9 in the chamber 6 in order to dispense the fluid product. Provided on the drive member 12 of the drive unit 3 in the transition between the sleeve-shaped front region and the rod 13 is a breaking point 26. Due to the forwardly directed force of the spring 23 and the abutment of the collar 22 on the webs 24 of the first housing part 1 during the forward stroke of the injection syringe 2, the breaking point 26 breaks as a result of the sudden restraining effect of the syringe on abutment. The rod 13 is pushed further inside the sleeve-shaped region of the drive member 12 in the forward stroke direction by the forwardly directed force of the spring 23 and thus also drives the plunger rod 8 in the forward stroke direction inside the syringe housing 21. As a result, the stopper 9 is moved in the forward stroke direction inside the chamber 6 and the fluid product is dispensed out of the chamber 6 through the injection needle 7.

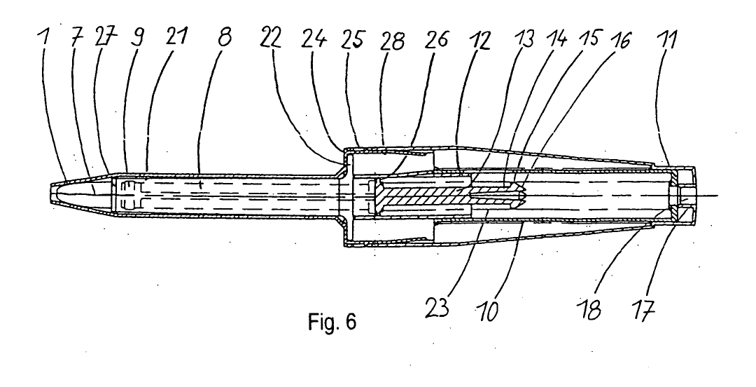

FIG. 6 illustrates the administering system with the needle in a retracted position, in which the injection needle 7 is retracted into the interior of the first housing part 1. The injection syringe 2 is provided with a retractor mechanism for retracting the injection needle. The retractor mechanism may be operated in such a way, for example, that the stopper 9 pushed forward by the plunger rod 8 lies against a release 27 at the end of the stroke so that the injection needle 7 is automatically retracted into the interior of the syringe housing 21 and hence into the interior of the first housing part 1.

The administering process with the administering system of the present invention is therefore complete. The administering process is effected by pushing the second housing part 4 relative to the first housing part 1, with the front end of the first housing part 1 placed on a tissue surface at an injection point, the administering system being held by the administrator by the second housing part 4, which is pushed or urged towards the tissue surface. The displacement releases the drive unit and the retractor mechanism. In other words, the injection needle 7 firstly pierces the tissue and then the plunger rod 8 is moved forwards for dispensing purposes and finally the injection needle 7 is retracted into the syringe housing 21. During the entire procedure, the injection needle 7 is protected against access. After administering the injectable product, the first housing part 1 and the second housing part 4 can be separated from one another and the injection syringe 2 removed. A new injection syringe 2 can then be assembled with the other components of the administering system so that the system is ready for administering again. However, any of the individual components or items comprising the administering system may also be designed as disposable parts.

It would be possible to use the modular principle of the system of the present invention with various components. For example, different types of injection syringes may be used, or different drive units which are advantageously already in the pre-tensioned state may be used. Generally, any suitable form of the components may be used selectively and interchangeably.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A system for administering an injectable product, comprising:
a) an injection syringe, from which the injectable product is administered through an injection needle,
b) a drive unit; and
c) a housing for accommodating the injection syringe and the drive unit, wherein
d) the drive unit drives the injection syringe in the longitudinal direction of the housing relative to the housing.

2. The administering system as claimed in claim 1, wherein the injection syringe, the drive unit and the housing are separate components.

3. The administering system as claimed in claim 1, further comprising a retractor mechanism for retracting the injection needle into the interior of the housing.

4. The administering system as claimed in claim 3, wherein the retractor mechanism is disposed in the injection syringe.

5. The administering system as claimed in claim 4, wherein the retractor mechanism retracts the injection needle into the interior of a syringe housing.

6. The administering system as claimed in claim 1, wherein the drive unit drives a drive member of the injection syringe relative to a syringe housing to administer the injectable product.

7. The administering system as claimed in claim 3, wherein the drive unit releases the retractor mechanism.

8. The administering system as claimed in claim 3, wherein the retractor mechanism can be released by a stop of the drive member after the injectable product has been administered.

9. The administering system as claimed in claim 3, further comprising a release mechanism for releasing the drive unit and the retractor mechanism.

10. The administering system as claimed in claim 9, wherein the release mechanism comprises two housing parts of the housing which can be displaced relative to one another.

11. A device for administering an injectable product, the device designed as a modular system having selectively changeable components and comprising an injection syringe component, a drive unit component, and a housing component for accommodating the injection syringe and the drive unit, wherein the drive unit moves the injection syringe relative to the housing and wherein said injection syringe component is selected from suitable injection syringe components.

12. The device according to claim 11, wherein said injection syringe component, drive unit component, and housing component are selected from suitable respective components.

13. An administering device comprising:
a housing comprising a first housing part and a second housing part;
a syringe; and
a drive unit, wherein the drive unit moves the injection syringe relative to the housing.

14. The administering device according to claim 13, wherein the first housing part has a generally cylindrical, sleeve-shaped region for accommodating the injection syringe and another region comprising a stop for the injection syringe.

15. The administering device according to claim 14, wherein the drive unit comprises:
a sleeve with two ends, one end open and the other having a terminal wall with a central orifice;
a drive member concentrically arranged in the sleeve and displaceable relative to the sleeve; and
a rod extending centrally along the longitudinal axis of the sleeve, said rod having one end that splits into two wings which have projections extending outwardly from the rod at their ends and oblique terminal surfaces converging to a point on the longitudinal axis, said wings defining a space whereby the wings can be bent radially inwardly, wherein end of the rod projects through the central orifice and the projections lie against the external face of the terminal wall, the drive member thereby not displaceable relative to the sleeve and held in a pre-tensioned state by a spring between the drive member and the terminal wall.

16. The administering device according to claim 15, wherein the second housing part is generally sleeve-shaped to accommodate the drive unit and has one end that adjoins a complementary region of the first housing part and a second end having a coaxially disposed, inwardly directed sleeve-shaped extension around a central opening.

17. The administering device according to claim 16, further comprising a locking cap at the second end of the second housing part, said cap comprising a pin extending through the opening of the second housing part.
